(19) United States
(12) Patent Application Publication
(10) Pub. No.: US 2007/0071810 A1
(43) Pub. Date: Mar. 29, 2007

(54) Additive for tablets

(76) Inventors: Kenichi Kudo, Yamatotakada-shi (JP); Mamoru Wada, Kitakatsuraqi-gun (JP); Michihiro Sunako, Uda-gun (JP); Junichi Takahara, Kashiba-shi (JP); Takeshi Takaha, Kobe-shi (JP); Kazutoshi Fujii, Suita-shi (JP); Takashi Kuriki, Suita-shi (JP)

Correspondence Address: MARK D. SARALINO (GENERAL);RENNER, OTTO, BOISSELLE & SKLAR, LLP, 1621 EUCLID AVENUE, NINETEENTH FLOOR, CLEVELAND, OH, 44115-2191, US

(21) Appl. No.: 10/569,582

(22) PCT Filed: Aug. 24, 2004
(86) PCT No.: PCT/JP04/12080

§ 371 Date: Aug. 22, 2006

(30) Foreign Application Priority Data
Aug. 26, 2003 (JP) 2003-301004

(51) Int. Cl. A61K 9/20; A61K 31/715; A61K 31/716

(52) U.S. Cl. 424/464; 514/54

(57) ABSTRACT

A disintegrant for tablets consisting of an α-1,4-glucan having a degree of polymerization of not less than 180 and less than 1230 and a dispersity (weight average molecular weight “Mw”/number average molecular weight “Mn”) of not more than 1.25 or a modified product thereof. A binder for tablets consisting of an α-1,4-glucan having a degree of polymerization of not less than 1230 and not more than 37000 and a dispersity of not more than 1.25, or a modified product thereof. A binding-disintegrating agent for tablets consisting of a low molecular weight α-1,4-glucan or a modified product thereof, and a high molecular weight α-1,4-glucan or a modified product thereof, wherein the low molecular weight α-1,4-glucan has a degree of polymerization of not less than 180 and less than 1230 and a dispersity of not more than 1.25, and wherein the high molecular weight α-1,4-glucan has a degree of polymerization of not less than 1230 and not less than 37000 and a dispersity of not more than 1.25.

### TECHNICAL FIELD

The present invention relates to an additive for tablets having improved disintegration or improved bondability and a tablet using the same.

### BACKGROUND ART

A tablet is one of the most useful forms of administration means for medicines, because it is easy to handle and allows for stable control of dosage with high accuracy. Tablets generally comprise various additives other than the principal bioactive agent in order to improve the properties of the tablets. Examples of these additives include an excipient, a disintegrant, a binder, a lubricant and the like.

An excipient is added to a tablet to give it form and to provide bulk. As the excipient, microcrystalline cellulose, lactose, starch and the like are generally used. However, when a tablet contains just one of those as an excipient, the tablet may have a longer disintegration time and thus the principal agent may not be absorbed quickly into the body, or the tablet may be damaged during packaging or transporting due to its reduced hardness. Therefore, in common tablets, two or more excipients are added, or a disintegrant or a binder is additionally added.

A disintegrant has the action to facilitate disintegration of a tablet in gastrointestinal fluids or the oral cavity. As the disintegrant, carmellose sodium, cross-povidone, partially pregeratinized starch and the like are generally used. Carmellose (CMC) is a typical disintegrant, which has short disintegration time, but has the disadvantage of pH susceptibility because it has a leaving group. The carmellose has additional disadvantages such as concerns about safety, because it is manufactured by chemical treatment, and poor shapability. Partially pregeratinized starch is a typical disintegrant of starch materials. It has the disadvantage that a large amount thereof adversly delays disintegration.

A binder is added in order to bind ingredient particles to each other and to increase hardness of a tablet upon production of the tablet. As the binder, hydroxypropylmethyl cellulose, gum arabic, gelatin and the like are generally used.

Generally, these additives for tablets often have not only one separate function per additive but multiple functions per substance such as shapability and disintegrating property, shapability and bondability, and the like.

Disintegration and hardness of a tablet are closely related. When a large amount of a disintegrant is added to shorten the disintegration time, a tablet has reduced hardness, and to the contrary, when a large amount of a binder is added, a tablet has increased hardness, but has inferior disintegration. No excipient has been known to achieve those two properties alone.

Furthermore, to make a tablet easily swallowable by reducing the size thereof, there is a need for an additive for tablets which exerts sufficient function in small amounts relative to the principal agent.

Starch and its derivatives are now used as additives for tablets. Natural starch is generally a mixture of amylose and amylopectin. Amylose is a polymer having a structure in which glucose residues are linearly connected via mainly α-1,4-glucoside bonds. Recent studies have shown that there are some branched parts in amylose. Amylopectin is a branched polymer having a structure in which glucose residues are linearly connected via α-1,4-glucoside bonds and branched glucoseresidues are connected therefrom via α-1,6-glucoside bonds.

Amylose is known to swell in the presence of water and to form a helical crystal by hydrogen bonds. Some additives for tablets taking advantage of such properties have been studied. Japanese Patent National Phase PCT Laid-Open Publication No. 10-506627 (Patent Document 1) describes an excipient which uses amylose obtained from natural starch. In Japanese Patent National Phase PCT Laid-Open Publication No. 8-507769 (Patent Document 2), cross-linked amylose is used as a binder and a disintegrant for tablets.

There are some known methods for obtaining amylose from natural starch. For example, there is a method of degrading a branched part of natural starch through the action of an enzyme (e.g., isoamylase or pullulanase, which are known to be debranching enzymes) that specifically cleaves α-1,6-glucoside bonds to obtain amylose (so called enzymatic starch degradation method). There is also a method of precipitating an amylose/butanol complex from starch paste to separate amylose.

However, the following problems with amyloses thus obtained from natural starch have been pointed out:
(a) amyloses contained in natural starch generally have a wide dispersity (Mw/Mn) of not less than 1.3. Such amyloses are a mixture of (i) low molecular weight amyloses with a high crystallinity index which are difficult to swell, (ii) high molecular weight amyloses of high bonding strength and (iii) amyloses of middle molecular weight between them which swell easily. Consequently, those amyloses of various molecular weights inhibit each other and counteract the excellent features of the other amyloses of different molecular weights. Therefore, it cannot exert a sufficient function of shapability to a tablet, as well as disintegrating property and bondability to a finished tablet;
(b) molecular weights of amyloses contained in natural starch are generally from a few dozen kDa to several hundred kDa, which is low; and
(c) separation of amyloses from natural starch is a complicated procedure and results in low yield, which cannot be applied to an industrial production method.

From the reasons described above, an application of amyloses obtained from natural starch to tablets would not be developed.

There have been some known methods of synthesizing α-1,4-glucan by connecting glucose residues by the action of an enzyme (enzymatic synthesizing methods).

For one example, there is a method of reacting an amylosucrase (EC 2.4.1.4) with sucrose as a substrate (hereinafter, abbreviated to the AMSU method). An α-1,4-glucan obtained by the AMSU method has a low degree of polymerization. It is reported that even when an α-1,4-glucan is produced by using a highly purified amylosucrase, it has a molecular weight of 8,941Da (Montalk et. al., FEBS Letters 471, pp 219-223 (2000); Non-patent Document 1).

Japanese Patent National Phase PCT Laid-Open Publication No. 2002-511429 (Patent Document 3) discloses a sustained release tablet using an α-1,4-glucan prepared by the AMSU method. In Patent Document 3, it is described that dispersity of 1.5 to 15 of the α-1,4-glucan is particularly preferred. This dispersity is equal to that of amylose derived from natural starch, and has a wide distribution of molecular weights. Accordingly, the α-1,4-glucan used in Patent Document 3 has no advantage over natural starch. Moreover, a sustained release tablet is in a totally different technical field than the purposed tablet of the present invention. As described in “BEST MODE FOR CARRYING OUT THE INVENTION” hereinafter, the tablet used in the present specification does not include a sustained release tablet. For the tablet of the present invention, it is preferable that the tablet is disintegrated immediately after oral administration (e.g., within a minute) and the principal agent in the tablet is released in the oral cavity or gastrointestine, and in contrast, the sustained release tablet is preferably disintegrated gradually and releases the principal agent over a long period of time after oral administration (e.g., not less than 24 hours).

If an α-1,4-glucan having small dispersity, i.e., a narrow distribution of molecular weights, is obtained by the AMSU method, the average molecular weight thereof is small, as described above. An α-1,4-glucan of a molecular weight of not more than several tens of thousands Da has very high crystallinity, and has very little shapability and bondability to other α-1,4-glucan powders or to other additives and drugs. Consequently, in the above application, only a small amount of the high molecular weight portion, which exists in a mixed state in α-1,4-glucans of high dispersity, i.e., an α-1,4-glucan having wide distribution of molecular weights, is thought to contribute to binding and sustained release of a tablet. An α-1,4-glucan obtained by the AMSU method essentially has no bondability and shapability, or very little if it has any.

As another method of enzymatic synthesis, there is a method using a glucan phosphorylase α-glucan phosphorylase, EC 2.4.1.1; generally referred to as a phosphorylase). These methods include a method of allowing the phosphorylase to act alone with a substrate (glucose-1-phosphate) to transfer a glucosyl group thereon to a primer (e.g., maltoheptaose) (called the GP method), and a method of synthesizing G-1-P from sucrose by using the phosphorylase and a sucrose phosphorylase and transferring a glucosyl group on the G-1-P to a primer (called the SP-GP method) (see, for example, International Publication No. WO 02/097107 Pamphlet (Patent Document 4)).

A method of using an α-1,4-glucan prepared by the GP method or SP-GP method for biodegradable articles is described in International Publication No. WO 02/06507 Pamphlet (Patent Document 5). In the pamphlet, for example, amylose of molecular weight 84.4 kDa and dispersity 1.02, as sample No. 3; amylose of molecular weight 110.0 kDa and dispersity 1.01, as sample No.4; amylose of molecular weight 276.1 kDa and dispersity 1.01, as sample No. 5; and amylose of molecular weight 741.9 kDa and dispersity 1.01, as sample No. 6 are described. This pamphlet describes that such an enzymatically synthesized amylose can be used as a matrix material for medicines, agricultural chemicals, fertilizers and the like, in which water soluble synthetic macromolecules, natural starch and proteins are conventionally used. However, “a matrix material for medicines, agricultural chemicals, fertilizers and the like” described in this pamphlet means a material that can be added in a large amount to medicines, agricultural chemicals, fertilizers and the like, has no specific function and only has a function of low importance such as the bulk effect of a filler. The matrix material has a function of low importance such as the bulk effect and very little effect on the properties of a tablet when added. In other words, the matrix material is required not to affect the properties of the tablet and the activity of a drug in the tablet when the matrix material is mixed with an active ingredient such as a drug or an additive having another function at any ratio. In contrast,a binder is added to a tablet containing an active ingredient which is difficult to form for the purpose of increasing the hardness of the tablet. A disintegrant is added to a tablet containing an ingredient difficult to disintegrate after oral administration, such as crude drugs, for the purpose of achieving quick release of the active ingredient. The hardness increasing effect or disintegration effect varies depending on the amount of binder or disintegrant added. Furthermore, to downsize a tablet or increase the proportion of an active ingredient in a tablet, there is a need for a binder and a disintegrant which can exert these effects in as small an amount as possible. Usage like this is contradictory to usage as a filler. Accordingly, a material having a special function such as a binder or a disintegrant is generally not included in “a matrix material”.

The pamphlet does not disclose or suggest a material having a special function such as a binder or a disintegrant. A binder is a material having an extremely special function, because the binder is a material added to a tablet containing an active ingredient which is difficult to form for the purpose of increasing the hardness of the tablet. A disintegrant is also a material having an extremely special function, because the disintegrant is a material added to a tablet containing an ingredient difficult to disintegrate after oral administration, such as crude drugs, in order to achieve quick release of the active ingredient. Furthermore, to downsize a tablet or increase the proportion of an active ingredient in a tablet, the binder and the disintegrant are required to exert effects in as small of an amount as possible. From these meanings, the binder and disintegrant are contradictory to a filler.

It is not easy for one skilled in the art to use the amylose described in the pamphlet as a material having a special function such as a binder or a disintegrant for tablets, because the matrix material described in the pamphlet generally means a material having no special function. From the pamphlet, it cannot be expected that an α-1,4-glucan of a specific degree of polymerization is superior as a disintegrant or a binder for tablets. This pamphlet describes, with regard to the degree of polymerization and properties of α-1,4-glucan, only that an α-1,4-glucan of a high degree of polymerization is water soluble and α-1,4-glucan of a low degree of polymerization has a gelling or crystallizing property, and does not describe or suggest the swelling property required for a disintegrant and the bondability required for a binder. Both swellability and bondability are properties not required for construction of the molded article described in the pamphlet.

Swellability and bondability are extremely special properties, which have been found and related only after studying the relationship between the properties of a tablet and the degree of polymerization of α-1,4-glucan during examination of application of α-1,4-glucan to tablets in the present invention. Therefore, the binder and the disintegrant of the present invention differ quite significantly from the matrix material described in the pamphlet.

It is not easy for one skilled in the art to use the amylose described in the pamphlet for tablets. Furthermore, it is also not easy to use these amyloses as a disintegrant or a binder for tablets, because the α-1,4-glucan used in the pamphlet is not commonly commercially available, and it is difficult to carry out an evaluation experiment itself. Those who intend to carry out an evaluation experiment must produce an enzyme and produce an α-1, 4-glucan with the enzyme by themselves. It is only then that evaluation of function as a binder by measuring hardness of tablets formed using an α-1,4-glucan of a specific degree of polymerization and evaluation of function as a disintegrant by measuring the degree of swelling of an α-1,4-glucan of a specific degree of polymerization are possible. As described above, the procedures for conducting the invention of the aforementioned pamphlet are complicated, but it goes without saying that there is sufficient description in the aforementioned pamphlet for conducting the invention of the aforementioned pamphlet.

Moreover, this pamphlet does not describe or suggest a combination of many amyloses of different molecular weights.
Patent Document 1: Japanese Patent National Phase PCT Laid-Open Publication No. 10-506627 (pp. 2-4)
Patent Document 2: Japanese Patent National Phase PCT Laid-Open Publication No. 8-507769 (p. 2)
Patent Document 3: Japanese Patent National Phase PCT Laid-Open Publication No. 2002-511429 (p. 2)
Patent document 4: International Publication No. WO02/097107 pamphlet (pp. 127-134)
Patent Document 5: International Publication No. WO 02/06507 pamphlet (pp. 22 and 23)
Non-patent Document 1: Montalk et. al., FEBS Letters 471, 2000, pp. 219-223

### DISCLOSURE OF THE INVENTION

### PROBLEMS TO BE SOLVED BY THE INVENTION

The present invention is intended to solve the aforementioned problems, and an object of the present of invention is to provide an additive for tablets that satisfies compression moldability as well as disintegrating property or strength.

### MEANS FOR SOLVING THE PROBLEMS

In order to solve the aforementioned problems, the present inventors continued to intensively study additives for tablets using an enzymatically synthesized α-1,4 -glucan, which has narrower dispersity compared with that of natural amylose and has a precisely controlled degree of polymerization and, as a result, finally found that disintegration and bondability are varied depending on the degree of polymerization of an α-1,4-glucan, which resulted in completion of the present invention. By using an enzymatically synthesized α-1,4-glucan in a tablet alone or in combination, an additive for tablets excellent in disintegration and bondability is provided.

A disintegrant for tablets of the present invention consists of an α-1,4-glucan having a degree of polymerization of not less than 180 and less than 1230 and a dispersity (weight average molecular weight “Mw”/number average molecular weight “Mn”) of not more than 1.25 or a modified product thereof.

In one embodiment, said α-1,4-glucan can be an enzymatically synthesized α-1,4-glucan.

In one embodiment, said disintegrant can be a modified product of the α-1,4-glucan, and said modification can be a chemical modification selected from the group consisting of esterification, etherification and cross-linking.

A binder for tablets of the present invention consists of an α-1,4-glucan having a degree of polymerization of not less than 1230 and not more than 3700 and a dispersity of not more than 1.25 or a modified product thereof.

In one embodiment, said α-1,4-glucan can be an enzymatically synthesized α-1,4-glucan.

In one embodiment, said binder can be a modified product of said α-1,4-glucan, and said modification can be a chemical modification product selected from the group consisting of esterification, etherification and cross-linking.

A binding-disintegrating agent for tablets of the present invention consists of a low molecular weight α-1,4-glucan or a modified product thereof, and a high molecular weight α-1,4-glucan or a modified product thereof, wherein said low molecular weight α-1,4-glucan has a degree of polymerization of not less than 180 and less than 1230 and a dispersity of not more than 1.25, and wherein said high molecular weight α-1,4-glucan has a degree of polymerization of not less than 1230 and less than 37000 and a dispersity of not more than 1.25.

In one embodiment, said α-1,4-glucan can be an enzymatically synthesized α-1,4-glucan.

In one embodiment, said binding-disintegrating agent can be a modified product of the α-1,4-glucan, and said modifications can be a chemical modification selected from the group consisting of esterification, etherification and cross-linking.

In one embodiment, the weight ratio of said low molecular weight α-1,4-glucan or a modified product thereof to said high molecular weight α-1,4-glucan or a modified product thereof can be 98:2 to 60:40.

In one embodiment, the weight ratio of said low molecular weight α-1,4-glucan or a modified product thereof to said high molecular weight α-1,4-glucan or a modified product thereof can be 2:98 to 40:60.

A tablet of the present invention contains a low molecular weight α-1,4-glucan or a modified product thereof, and a high molecular weight α-1,4-glucan or a modified product thereof, wherein said low molecular weight α-1,4-glucan has a degree of polymerization of not less than 180 and less than 1230 and a dispersity of not more than 1.25, and wherein the high molecular weight α-1,4-glucan has a degree of polymerization of not less than 1230 and less than 37000 and a dispersity of not more than 1.25.

### EFFECTS OF THE INVENTION

By appropriately changing the molecular weight of an α-1,4-glucan having small dispersity contained in a tablet, the disintegration time and bondability of the tablet can be controlled as desired. According to the present invention, an additive for tablets satisfying the desired disintegration and hardness can be provided.

### BEST MODE FOR CARRYING OUT THE INVENTION

(Definition of Terms)

(Dispersity, Mw/Mn)

Macromolecular compounds do not have a single molecular weight but have a certain variation in molecular weight regardless of whether it is naturally-occurring or not, except for special cases such as protein. Therefore, to represent a degree of dispersion of molecular weight of macromolecular compounds, dispersity, Mw/Mn, is generally used in the field of macromolecular chemistry. Dispersity, Mw/Mn, is represented as a ratio of weight average molecular weight “Mw” to number average molecular weight “Mn” (i.e., Mw/Mn). Dispersity is an indicator of the breadth of the molecular weight distribution of macromolecular compounds. When a macromolecular compound has a single molecular weight, Mw/Mn is equal to 1, and when a macromolecular compound has a wider molecular weight distribution, the macromolecular compound has a lager Mw/Mn than 1. The term “molecular weight”as used herein, refers a weight average molecular weight unless otherwise stated.

The term “tablet”, as used herein, refers to a tablet produced by compressing tablet materials containing an active ingredient into a certain shape which disintegrate within 10 minutes after oral administration. In other words, the concept of the “tablet” herein does not include a “sustained release tablet”. Such a tablet is also called a compression tablet after its production method. In the present specification, a tablet may be a tablet for pharmaceutical applications, or may be a tablet for food applications. Examples of the tablet for food applications include a tablet snack and a health food tablet (e.g., a supplement).

The term “disintegrant”, as used herein, refers to an additive imparting disintegration ability in water or gastric juice to a tablet.

The term “binder”, as used herein, refers to an additive used for producing a stable tablet or granule by imparting binding force to a mixture of ingredient powders.

The term “binding-disintegrating agent”, as used herein, refers an additive exerting both actions as a disintegrant and as a binder.

The term “α-1,4-glucan”, as used herein, refers a saccharide having structural units of D-glucose, which contains two or more saccharide units connected via only α-1,4-glucoside bonds. An α-1,4-glucan is a linear molecule. An α-1,4-glucan is also called as a linear glucan. The number of saccharide units contained in one α-1,4-glucan molecule is referred to as the degree of polymerization.

An α-1,4-glucan used in the present invention can be prepared by known methods in the art. Preferably, it is prepared by a known enzymatic synthesizing method. Examples of such enzymatic synthesizing methods include a method using a glucan phosphorylase (a-glucan phosphorylase, EC 2.4.1.1; generally referred to as a phosphorylase). The phosphorylase is an enzyme catalyzing a phosphorolysis reaction.

As an example of an enzymatic synthesizing method using phosphorylase, there is a method of allowing action of the phosphorylase with a substrate glucose-1-phosphate (hereinafter, referred to as G-1-P) to transfer a glucosyl group thereon to a primer (e.g., maltoheptaose) (hereinafter, referred to as the GP method). Although the GP method is costly for industrially producing α-1,4-glucan, due to the high expense of G-1-P as a raw material, the method has the remarkable advantage that an absolutely linear α-1,4-glucan can be obtained by sequentially connecting each saccharide unit with a α-1,4-glucoside bond alone. The GP method is known in the art.

Another example of an enzymatic synthesizing method using phosphorylase is a method of enzymatically synthesizing α-1,4-glucan by using sucrose as a substrate and, for example, maltooligosaccharide as a primer, and allowing simultaneous reaction of the sucrose phosphorylase (EC2.4.1.7) and the phosphorylase in the presence of inorganic phosphoric acid (hereinafter, referred to as the SP-GP method). The SP-GP method has advantages that the α-1,4-glucan produced is absolutely linear, as in the GP method, the molecular weight can be controlled as desired, and the production costs are less because inexpensive sucrose is used as the raw material. The SP-GP method is known in the art. An efficient production method by the SP-GP method is described in, for example, International Publication No. WO 02/097107 pamphlet. An α-1,4-glucan used in the present invention can be produced according to the method described in this pamphlet.

On the other, the above-described AMSU method is also a method of synthesizing an α-1,4-glucan using an enzyme, but not suitable for use in the present invention, because an obtained α-1,4-glucan has a very low degree of polymerization (about 9 kDa or less of molecular weight).

An enzymatically synthesized α-1,4-glucan which is enzymatically synthesized by employing the aforementioned GP method and/or the aforementioned SP-GP method has the following characteristics:
(i) narrow molecular weight distribution (not more than 1.1 of Mw/Mn);
(ii) those having any degree of polymerization (about 60 to about 37000) can be obtained by controlling the production conditions appropriately;
(iii) absolutely linear and no branched structure found slightly in amylose fractionated from natural starch;
(iv) similarly to natural starch, being constructed from glucose residues only and thus all of the α-1,4-glucan, a degradation intermediate thereof and a final degradated product have no toxicity to a living body; and
(v) similarly to starch, being able to be chemically modified as necessary.

The enzymatically synthesized α-1,4-glucans which are enzymatically synthesized by employing the aforementioned GP method and/or the aforementioned SP-GP method have various molecular weights and crystal forms according to production conditions. In the production of α-1,4-glucan by the GP method or the SP-GP method, conditions for synthesizing a low molecular weight α-1,4-glucan make the resultant α-1,4-glucan precipitate in the reaction solution, and on the contrary, conditions for synthesizing a high molecular weight α-1,4-glucan allow the resultant α-1,4-glucan to remain in a dissolved state. The boundary between them,which may be varied depending on synthesizing conditions, is generally a degree of polymerization of about 1230 (about 200 kDa of molecular weight). The difference in state at production results in different crystal forms of α-1,4-glucans, and as listed in Table 1 below, results in different properties when used in a tablet. Not only the molecular weight, but the crystal form affects the properties. When an amylose is separated from natural starch, such control of molecular weight and crystal form of the resultant amylose will be impossible.

TABLE 1

| | | |
|---|---|---|
| degree of | less than 1230 | not less than 1230 |
| polymerization | (200 kDa) | (200 kDa) |
| (molecular weight) | | |
| state at | precipitated | solution |
| production | | |
| crystal form | retrograded + | V-form and/or amorphous |
| | amorphous | |
| property | swelling by contacting | by contacting with water, |
| | with water, but not | easily resulting in |
| | resulting in bonding | bonding between |
| | between α-1,4- | α-1,4-glucan molecules |
| | glucan molecules | |
| use in a tablet | disintegrant | binder |
| | (excipient) | |

The above-described classification into two types is just for convenience in describing the properties based on the molecular weight and the crystal form of the enzymatically synthesized α-1,4-glucan. In the boundary region between these two types, an enzymatically synthesized α-1,4-glucan has both disintegration ability and bondability, and a balance of these is varied continuously in accordance with the molecular weight. Therefore, a user can select and use an enzymatically synthesized α-1,4-glucan having an arbitrary molecular weight according to the kind of principal agent and the required property for the tablet. Two or more enzymatically synthesized α-1,4-glucans having different molecular weight may be used as a mixture.

Amyloses obtained from natural starch and amyloses obtained by the AMSU method that contain a small amount of a high molecular weight amylose having binding action and shaping action have wide molecular weight distributions, and thus can be regarded as a mixture of various amyloses having different molecular weights. However, those distributions thereof are primarily dependent on origins and preparation methods, and properties when used in tablets are fixed. In contrast, the mixture of α-1,4-glucans used in the present invention has a narrow molecular weight distribution, and its properties are determined by mixing amyloses which differ according to their molecular weights, and therefore the present mixture is obviously different from the prior art in the point that a property suitable for an intended tablet can be easily achieved by changing the mixing ratio.

The term “low molecular weight α-1,4-glucan”, as used herein, refers to an α-1,4-glucan having a degree of polymerization of less than 1230.

The term “high molecular weight α-1,4-glucan”, as used herein, refers to an α-1,4-glucan having a degree of polymerization of not less than 1230.

The term “modification”, as used herein, refers to a product obtained by chemically modifying a subject. Examples of the modified product include esterification, etherification and cross-linking.

Esterification may be carried out by, for example, reacting an α-1,4-glucan with an esterifying reagent (e.g., acid anhydride, organic acid, acid chloride, ketene, and other esterifying reagents) in various solvents or without solvent. By such esterification, for example, an acylated ester such as acetate and propionate is obtained.

Etherification may be carried out by, for example, reacting an α-1,4-glucan with an etherifying agent (e.g., alkyl halide, dialkyl sulfate) in the presence of alkaline. By such etherification, for example, carboxymethyl ether, hydroxypropyl ether, hydroxymethyl ether, methyl ether or ethyl ether is obtained.

Cross-linking may be carried out by, for example, reacting an α-1,4-glucan with a cross-linking agent (e.g., formalin, epichlorohydrin, glutaraldehyde, various diglycidyl ethers, various esters).

(1. Disintegrant For Tablets)

A disintegrant for tablets of the present invention consists of α-1,4-glucans having a degree of polymerization of not less than 180 and less than 1230 and a dispersity (weight average molecular weight “Mw”/number average molecular weight “Mn”) of not more than 1.25, or a modified product thereof. A degree of polymerization of an α-1,4-glucan used as the disintegrant for tablets is more preferably not less than 200 and less than 1200, more preferably not less than 400 and less than 1100, and still more preferably not less than 600 and less than 900.

A disintegrant makes a tablet disintegrate by swelling with water. The degree of swelling is an indicator of the ease of disintegration (disintegration ability) of a certain substance. Swelling is the phenomenon of a cross-linked macromolecular solid considerably enlarging its volume by absorbing a large amount of liquid when it is immersed in the liquid. Cases of absorbing a small amount of liquid and of absorbing liquid infinitely to lose solid properties are not referred to as “swelling”, but as “absorbing” and “dissolving” respectively. The higher the degree of swelling, the higher the disintegration ability is. The degree of swelling can be measured by a known method in the art, for example, an experiment for measuring the degree of swelling will be described herein. In the present specification, the degree of swelling refers to the volume obtained by dispersing 5 g of α-1,4-glucan into 60 ml of distilled water, adding distilled water to obtain 100 ml of mixture in 100 ml measuring cylinder, allowing the mixture to stand overnight, then measuring the volume of precipitates and converting the volume of precipitates into the volume per 1 g. An α-1,4-glucan having a degree of swelling of not less than 5 ml/g can be used as a disintegrant. The degree of swelling of the disintegrant of the present invention is preferably not less than 5 ml/g, and more preferably 10 ml/g. The degree of swelling is not specifically upper limited, but may be appropriately set to not more than 50 ml/g, not more than 40 ml/g, not more than 30 ml/g, or not more than 20 ml/g as necessary. A degree of swelling that is too high can result in a tablet having an extremely low hardness.

The α-1,4-glucan having a degree of polymerization of not less than 180 and less than 1230 and a dispersity (weight average molecular weight “Mw”/number average molecular weight “Mn”) of not more than 1.25, or a modified product thereof used in the present invention can be prepared by a known method in the art. It is prepared preferably by a known enzymatic synthesizing method, more preferably by the GP method or the SP-GP method, and more preferably by the SP-GP method.

A disintegrant used in the present invention may be an unmodified α-1,4-glucan, or may be a modified product of an α-1,4-glucan. When the disintegrant is a modified product of an α-1,4-glucan, the modification is preferably a chemical modification selected from the group consisting of esterification, etherification and cross-linking, more preferably etherification, and most preferably carboxymethylation. Chemical modifications such as carboxymethylation have the advantage of enhanced disintegration. By applying such a chemical modification alone or in combination, the hydrophilicity, hydrophobicity, water-solubility, viscosity and the like of an α-1,4-glucan can be changed. According to the kind of major ingredient and the required properties of the tablet, an α-1,4-glucan modified by those chemical modifications can be selected.

(2. Binder For Tablets)

A binder for tablets of the present invention consists of an α-1,4-glucan having a degree of polymerization of not less than 1230 and not more than 37000 and a dispersity of not more than 1.25, or a modified product thereof. Although an α-1,4-glucan having a degree of polymerization of more than about 37000 has an effect as a binder, from the point of yield by the SP-GP method, an α-1,4-glucan having a degree of polymerization of not more than about 37000 is preferable, and an α-1,4-glucan having a degree of polymerization of not more than about 18600 is more preferable. The degree of polymerization of an α-1,4-glucan used as a binder for tablets is more preferably not less than 1500 and not more than 30000, and more preferably not less than 1600 and not more than 18000.

A binder increases the hardness of a tablet by binding substances contained in the tablet. The hardness of a tablet obtained by tableting a certain substance is only an indicator of its property as a binder of the substance. The higher the hardness is, the stronger the binding force is. When a tablet is prepared in the same conditions as of “performance test as a binder” described in the Examples using a binder of the present invention, the tablet preferably has a hardness of not less than 8 kgf, more preferably has a hardness of not less than 10 kgf, and more preferably has a hardness of not less than 11 kgf. The hardness is not specifically upper limited, but may be appropriately set to not more than 30 kgf, not more than 25 kgf, not more than 20 kgf or not more than 16 kgf as necessary. A hardness that is too high can result in a tablet having extremely low disintegration.

The α-1,4-glucan having a degree of polymerization of not less than 1230 and not more than 37000 and a dispersity (weight average molecular weight “Mw”/number average molecular weight “Mn”) of not more than 1.25, or a modified product thereof used in the present invention can be prepared by a known method in the art. It is prepared preferably by a known enzymatic synthesizing method, more preferably by the GP method or the SP-GP method, and more preferably by the SP-GP method.

A binder used in the present invention may be an unmodified α-1,4-glucan, or may be a modified product of an α-1,4-glucan. When the binder is a modified product of an α-1,4-glucan, the modification is preferably a chemical modification selected from the group consisting of esterification, etherification and cross-linking, more preferably cross-linking or etherification, and most preferably cross-linking. Chemical modifications such as cross-linking have the advantage of enhanced bondability without exceedingly degrading the disintegration. By applying such a chemical modification alone or in combination, the hydrophilicity, hydrophobicity, water-solubility, viscosity and the like of the α-1,4-glucan can be changed. According to the kind of major ingredient and the required properties of the tablet, an α-1,4-glucan modified by those chemical modifications can be selected.

(3. Binding-Disintegrating Agent For Tablets)

A binding-disintegrating agent for tablets of the present invention consists of a low molecular weight α-1,4-glucan or a modified product thereof, and a high molecular weight glucan or a modified product thereof.

The low molecular weight α-1,4-glucan contained in the binding-disintegrating agent for tablets of the present invention has a degree of polymerization of not less than 180 and less than 1230 and a dispersity of not more than 1.25.

The high molecular weight α-1,4-glucan contained in the binding-disintegrating agent for tablets of the present invention has a degree of polymerization of not less than 1230 and less than 37000 and a dispersity of not more than 1.25.

Those low molecular weight α-1,4-glucan and high molecular weight α-1,4-glucan are preferably enzymatically synthesized α-1,4-glucans.

A binding-disintegrating agent for tablets of the present invention may be an unmodified α-1,4-glucan, or may be a modified product of an α-1,4-glucan. When a binding-disintegrating agent is a modified product of an α-1,4-glucan, the modification is preferably a chemical modification selected from the group consisting of esterification, etherification and cross-linking, more preferably etherification, and most preferably carboxymethylation. Chemical modifications such as carboxymethylation have an advantage of enhanced disintegration. When the bondability of a binding-disintegrating agent is desired to be enhanced, chemical modifications such as cross-linking and etherification may be used. By applying such a chemical modification alone or in combination, the hydrophilicity, hydrophobicity, water-solubility, viscosity and the like of the α-1,4-glucan can be changed. According to the kind of major ingredient and the required properties of the tablet, an α-1,4-glucan modified by those chemical modifications can be selected.

A binding-disintegrating agent for tablets of the present invention can be produced by mixing a low molecular weight α-1,4-glucan having a specific weight average degree of polymerization and a dispersity of not more than 1.25, and a high molecular weight α-1,4- glucan having a specific weight average degree of polymerization and a dispersity of not more than 1.25. A binding-disintegrating agent for tablets of the present invention is produced by mixing two or more α-1,4-glucans. A binding-disintegrating agent for tablets of the present invention may be produced by mixing a plurality of, for example, 3 or more, 4 or more, 5 or more, 6 or more, 7 or more, 8 or more, 9 or more, 10 or more and the like of α-1,4-glucans. However, when too many kinds of α-1,4-glucans are mixed, they can interfere with the properties of each other. Therefore, the number of kinds of α-1,4-glucans thus mixed are preferably not more than 5, more preferably not more than 4, still more preferably not more than 3, and most preferably not more than 2. In the present specification, a single kind of α-1,4-glucan means that when about 300 μg of the α-1,4-glucan is subjected to gel filtration chromatography according to the method described in measuring methods section of Examples, the number of distinguishing peaks appearing is 1.

In one embodiment, in a binding-disintegrating agent for tablets of the present invention, the weight ratio of a low molecular weight α-1,4-glucan or a modified product thereof to a high molecular weight αa-1,4-glucan or a modified product thereof is preferably 98:2 to 60:40. When mixed in such a ratio, the properties as a disintegrant are strong.

In one embodiment, in a binding-disintegrating agent for tablets of the present invention, the weight ratio of a low molecular weight α-1,4-glucan or a modified product thereof to a high molecular weight α-1,4-glucanoramodified product thereof is 2:98 to 40:60. Whenmixed in such a ratio, the properties as a binder are strong.

The degree of swelling of a binding-disintegrating agent for tablets of the present invention is preferably not less than 5 ml/g, and more preferably not less than 10 ml/g. The degree of swelling is not specifically upper limited but may appropriately be set to not more than 50 ml/g, not more than 40 ml/g, not more than 30 ml/g, or not more than 20 ml/g as necessary. A degree of swelling that is too high can result in a tablet having an extremely low hardness.

When a tablet is prepared in the same conditions as of “performance test as a binder” described in the Examples using a binding-disintegrating agent for tablets of the present invention, the tablet preferably has a hardness of not less than 8 kgf, more preferably has a hardness of not less than 10 kgf, and more preferably has a hardness of not less than 11 kgf. The hardness is not specifically upper limited, but may be appropriately set to not more than 30 kgf, not more than 25 kgf, not more than 20 kgf or not more than 16 kgf as necessary. A hardness that is too high can result in a tablet having extremely low disintegration.

(4. Tablet)

A tablet of the present invention contains a low molecular weight α-1,4-glucan or a modified product thereof, and a high molecular weight glucan or a modified product thereof, wherein the low molecular weight α-1,4-glucan has a degree of polymerization of not less than 180 and less than 1230 and a dispersity of not more than 1.25, and the high molecular weight α-1,4-glucan has a degree of polymerization of not less than 1230 and less than 37000 and a dispersity of not more than 1.25. These low molecular weight α-1,4-glucans and high molecular weight α-1,4-glucans are as described in the above 1, 2 and 3.

A tablet of the present invention contains a principal agent. To the tablet of the present invention, additives can be added as necessary, other than the low molecular weight α-1,4-glucan and the high molecular weight α-1,4-glucan, such as an excipient, a disintegrant, a binder, a lubricant, a glidant and a coating agent, for the purpose of improving the properties of the tablet. Those additives are well known in the art, and described, for example, in Pharmacopeia of Japan.

A tablet of the present invention can be produced by utilizing techniques and facilities conventionally used for production of tablets without change. For example, those methods can be employed, such as the method of mixing and directly tableting a principal agent and additives and the method of granulating ingredients in a wet or dry process and then tableting.

The additives of the present invention (a disintegrant, a binder and a binding-disintegrating agent) can be used not only for medicinal tablets, but also as an excipient, a disintegrant and a binder for consolidating and using a powder active ingredient in the field of pesticides, fertilizers, cosmetics, foods, feed and the like.

The disintegration time of a tablet of the present invention is preferably not more than about 10 minutes, more preferably not more than about 5 minutes, still more preferably not more than about 3 minutes, even more preferably not more than about 1 minute, even still more preferably not more than about 40 seconds, especially preferably not more than about 30 seconds, and most preferably not more than about 25 seconds, when measured according to the method described in Examples 2 or 3 hereinbelow. A shorter disintegration time is more preferable.

The hardness of a tablet of the present invention is preferably not less than about 4.0, more preferably not less than about 4.5, and most preferably not less than about 5.0, when measured according to the method described in Example 2 hereinbelow. A tablet having a hardness that is too low can be disintegrated during transport. The hardness of a tablet is not specifically upper limited, but a hardness of, for example, up to 10.0, up to 9.0, or up to 8.0 may be preferably selected.

### EXAMPLES

Hereinbelow, the present invention is further described in detail by referencing Examples and Test Examples, but is not limited by those Examples and Test Examples.

(Preparation Method and Measuring Method)

In the Test Examples, a preparation method of a purified glucan phosphorylase derived from a potato tuber, a preparation method of a sucrose phosphorylase derived from Streptococcus mutans, a calculation method of a yield (%) of an α-1,4-glucan and a measuring method of a weight average molecular weight (Mw) and a number average molecular weight (Mn) were performed according to methods described in Japanese Patent Laid-open Publication No. 2002-345458 and International Publication No. WO 02/097107 pamphlet.

Specifically, the molecular weight of a synthesized α-1,4-glucan was measured as follows. Firstly, a synthesized glucan was completely resolved in 1N sodium hydroxide and neutralized with an appropriate amount of hydrochloric acid to obtain a mixture. The mixture containing about 300 μg of glucan was subjected to gel filtration chromatography accompanied with a differential refractometer and a multiple-angle light scattering detector to determine the weight average molecular weight. In detail, Shodex SB806M-HQ (manufactured by Showa Denko K.K) was used as the column, and the multiple-angle light scattering detector (DAWN-DSP, manufactured by Wyatt Technology Corporation) and differential refractometer (Shodex RI-71, manufactured by Showa Denko K. K) as detectors were connected in this order to use. The column was maintained at 40° C., and 0.1 M sodium nitrate solution was used as the eluent at a flow rate of 1 mL/min. The resultant signals were collected using data analysis software (trade name: ASTRA, manufactured by Wyatt Technology Corporation), and analyzed by the software to determine the weight average molecular weight and a number average molecular weight.

(Disintegration Test)

A disintegration test was carried out according to the method described in Pharmacopeia of Japan. For each sample, 6 tablets were measured for the disintegration time and an average was calculated from those.

(Hardness)

As for hardness, the load resulting in tablet destruction when loading in the direction of the diameter of the tablet was measured for 5 tablets of each sample by a Kiya hardness tester, and an average was calculated from those.

(Friability)

The friability of a tablet was determined as follows. 6.5 g of tablets was weighed and charged into a tablet friability tester, and the tablet friability tester was rotated lOO times at a speed of 25-rounds per minute. After the rotation ended, the tablets were taken and passed through a 10-mesh sieve to remove those powdered, and then the remaining tablets were weighed. A friability (%) is obtained by the following formula.
friability (%)={[(weight of tablets before test)−(weight of tablets after rotation ended)]/(weight of tablets before test)}×100 [formula 1]

### Test Example 1

Synthesis of α-1,4-Glucan

To 1 liter each of reaction mixtures containing 6 mM phosphate buffer (pH .7.0), 106 mM sucrose and a maltooligosaccharide mixture in various concentrations (2200,880, 176, 132, 44 or 8.8 mg/l, respectively) was added 1 unit/ml of purified glucan phosphorylase derived from a potato tuber prepared according to the method described in International Publication No. WO 02/097107 pamphlet and 1 unit/ml of sucrose phosphorylase derived from Streptococcus mutans prepared according to the method described in International Publication No. WO 02/097107 pamphlet, and kept at 37° C. for 16 hours. After the reaction end, the yield (%) ,weight average molecular weight (Mw) and molecular weight distribution (Mw/Mn) of the produced α-1,4-glucan were determined. Respective results are shown in Table 2 below.

TABLE 2

| | maltooligo- | | | | |
|---|---|---|---|---|---|
| | saccharide | yield | Mw | degree of | |
| sample | mixture (mg/l) | (%) | (kDa) | polymerization | Mw/Mn |
| 1 | 2200 | 95.6 | 11.0 | 70 | 1.04 |
| 2 | 880 | 90.2 | 30.2 | 186 | 1.03 |
| 3 | 176 | 91.0 | 89.4 | 552 | 1.02 |
| 4 | 132 | 87.9 | 108.2 | 668 | 1.02 |
| 5 | 44 | 85.7 | 281.0 | 1735 | 1.01 |
| 6 | 8.8 | 84.0 | 780.5 | 4818 | 1.01 |

According to Table 2, by changing the concentration ratio of a sucrose and a primer (i.e., maltooligosaccharide mixture), α-1,4-glucans of degrees of polymerization from 68 to 4818 (Mw 11.0 to 780.5kDa) were obtained. In those samples, the molecular weight distribution (Mw/Mn) of any α-1,4-glucan was narrow (for all, not more than 1.05). For those samples, the low molecular weight α-1,4-glucan such as those in samples 1 and 2 was precipitated. But for samples 3, 4, 5 and 6, the higher the degree of polymerization, the less precipitate was formed, and in samples 4 to 6, the reaction mixture was still transparent and the resultant α-1,4-glucan was water soluble.

### Test Example 2

Fractionation of Amylose of Natural Origin

30 g of starch separated from a potato tuber was added to 1 liter of hot water with stirring well to prepare a 3% paste. The paste was treated with an autoclave (120° C., for 30 minutes), and then undissolved residue was removed by a glass filter. To the resultant solution was added butanol (not less than a saturated amount, 15%), and the mixture was heated at 95° C. (in a boiling water bath) for 30 minutes, and then cooled slowly in a thermos bottle. After 1 day, precipitates (amylose/butanol complex) were centrifuged. Butanol was added to the separated complex once again and re-subjected to the same operation, and an amylose/butanol complex was precipitated from hot saturated butanol aqueous solution and separated by centrifugation. The separated amylose/butanol complex was washed with ethanol twice, dried under vacuum, and then passed through a 200-mesh sieve to give white powder. For the resultant amylose, the yield was 3.5 g, Mw was 280,000, and Mw/Mn was 3.3.

(Measuring Experiment for Degree of Swelling)

Degrees of swelling of the various α-1,4-glucans obtained in aforementioned Test Example 1 were measured. Firstly, to 60 ml of distilled water was dispersed 5 g of either α-1,4-glucan of 1 to 6 in Test Example 1, and adding distilled water to obtain 100 ml of mixture in 100 ml measuring cylinder, and allowed to stand overnight. The volume of precipitates was then read. By dividing the volume by 5, a volume of precipitates per 1 g of starting α-1,4-glucan (i.e., a degree of swelling) was calculated. Results are shown in Table 3 below.

TABLE 3

| sample | degree of swelling (ml/g) |
|---|---|
| 1 | 3.5 |
| 2 | 6.2 |
| 3 | 11.8 |
| 4 | 18.0 |
| 5 | — |
| 6 | 0 |

From the results, it was seen that samples 2 to 4 have a preferable degree of swelling as a disintegrant. Sample 5 could not be dispersed in water, because it turned into a gel when added to water. Further, sample 6 had a degree of swelling of 0, because it is dissolved absolutely. Therefore, it was shown that an α-1, 4-glucan having a degree of polymerization of 186 to 668 is preferable as a disintegrant.

(Test for Performance as a Binder)

Each of the various α-1,4-glucans obtained in aforementioned Test Example 1 was placed in a tableting mold (9mm of diameter, 5.5mm of thickness) alone, and a 500 kgf load was applied for 30 seconds by a desktop pressing machine (manufactured by Shimadzu Corporation; SSP-10A). Then, the load was released and the tablet was removed. The hardness of the resultant tablet was measured according to the method described above. Results are shown in Table 4 below.

TABLE 4

| sample | hardness (kgf) |
|---|---|
| 1 | 3.6 |
| 2 | 5.1 |
| 3 | 6.0 |
| 4 | 7.2 |
| 5 | 12.0 |
| 6 | 14.8 |

From the results, it was seen that an α-1,4-glucan having a larger degree of polymerization resulted in a higher hardness, and the α-1,4-glucans of samples 5 and 6 resulted in much larger hardnesses than the other α-1,4-glucans.

### Example 1

Utilization of a Low Molecular Weight α-1,4-Glucan As A Disintegrant

The α-1,4-glucan (sample 3) obtained in Test Example 1 was mixed with other ingredients for a tablet so that the tablet has a composition ratio shown in Table 5 below. The mixture was placed in a tableting mold (9 mm of diameter, 5.5 mm of thickness), and 500 kgf loads was applied for 30 seconds by a desktop pressing machine (manufactured by Shimadzu Corporation; SSP-10A). Then, the load was released and the tablet was removed.

TABLE 5

| ingredient | parts by weight |
|---|---|
| acetaminophen | 400 parts |
| Avicel (AvicelPH-101, manufactured | 300 parts |
| by Asahi Kasei Corporation) | |
| Pharmacopoeia lactose | 245 parts |
| α-1,4-glucan (sample 3) | 50 parts |
| magnesium stearate | 5 parts |
| total | 1000 parts |

### Comparative Example 1

Comparison with other Disintegrants

Tablets were obtained by a similar method as of Example 1, except that an α-1,4-glucan having a weight average molecular weight of 11 kDa (sample 1), partially pregeratinized starch (PC-10, manufactured by Asahi Kasei Corporation, sample name A), carmellose (NS-300, manufactured by Gotoku Chemical Company Ltd., sample name B) and microcrystalline cellulose (Avicel PH-101, manufactured by Asahi Kasei Corporation sample name C) were used respectively, instead of the α-1,4-glucan of sample 3. Avicel is a control.

### Example 2

Measurement of Disintegration Time in Distilled Water and Hardness

The disintegration time in distilled water and the hardness of each of the tablets in Example 1 and Comparative Example 1 were measured. Results are shown in Table 6 below.

TABLE 6

| | | | | disintegration | |
|---|---|---|---|---|---|
| | | degree of | Mw | time | hardness |
| | sample | polymerization | (kDa) | (second) | (kgf) |
| Example | 3(α-1,4-glucan) | 552 | 89.4 | 20 | 5.2 |
| Comparative | 1(α-1,4-glucan) | 68 | 11.0 | 26 | 3.1 |
| Example | A(partially | — | — | 27 | 3.8 |
| | pregeratinized | | | | |
| | starch) | | | | |
| | B(carmellose) | — | — | 24 | 5.0 |
| | C(microcrystalline | — | — | 35 | 5.3 |
| | cellulose) | | | | |

From the results, the tablet with the α-1,4-glucan of sample 3 added was disintegrated most rapidly. For the α-1,4-glucan having this degree of polymerization, the disintegration rate was considerably increased, but the hardness was slightly decreased.

On the other hand, the tablets with other ingredients added also had a shorter disintegration time, compared with the tablet with microcrystalline cellulose added (sample C), which is an excipient. However, the tablet with the α-1,4-glucan of sample 1 added having a smaller degree of polymerization or partially pregeratinized starch had a considerably decreased hardness.

As described above, the α-1,4-glucan of the present invention exhibits excellent suitability as a disintegrant.

### Example 3

Measurement of Disintegration Time In 1st Fluid of Japanese Pharmacopoeia and 2nd Fluid of Japanese Pharmacopoeia Assumed as Gastric or Intestinal Juices

Disintegration tests of tablets in Example 1 and Comparative Example 1 were carried out by using 1st fluid of Japanese Pharmacopoeia First Liquid (approximately pH 1.2) and 2nd fluid of Japanese Pharmacopoeia (approximately pH 6.8) instead of distilled water. Disintegration times are shown in Table 7 below.

TABLE 7

| | | disintegration time (second) | | |
|---|---|---|---|---|
| | | dis- | 1st fluid of | 2nd fluid of |
| | | tilled | Japanese | Japanese |
| | sample | water | Pharmacopoeia | Pharmacopoeia |
| Example | 3(α-1,4-glucan) | 20 | 21 | 20 |
| Compar- | 1(α-1,4-glucan) | 26 | 26 | 25 |
| ative | A(partially | 27 | 29 | 28 |
| Example | pregeratinized | | | |
| | starch) | | | |
| | B(carmellose) | 24 | 29 | 33 |
| | C(microcrystalline | 35 | 34 | 38 |
| | cellulose) | | | |

The disintegration time of the tablet with the α-1,4-glucan in Example 1 was almost unchanged by pH alteration, but for the tablet with carmellose of Comparative the Examples, delay of disintegration occurred in circumstances of low pH and high pH compared to in distilled water.

From the results, the disintegrating property of an α-1,4-glucan was found not to be affected by pH change.

### Example 4

Relationship Between an Amount Added and Disintegration Time

Tablets were prepared by mixing sample 3 in Test Example 1, partially pregeratinized starch (PC-10, manufactured by Asahi Kasei Corporation, sample name A) or carmellose (NS-300, manufactured by Gotoku Chemical Company Ltd., sample name B) with microcrystalline cellulose (Avicel PH-101) in various ratios and according to the method of Example 1, and measured for disintegration time in distilled water. Results are shown in Table 8.

TABLE 8

| | | weight ratio of a sample in a tablet/ | | | |
|---|---|---|---|---|---|
| | | disintegration time (second) | | | |
| | sample | 25% | 50% | 75% | 100% |
| Example | 3(α-1,4-glucan) | 24 | 21 | 19 | 18 |
| Comparative | A(partially | 28 | 28 | 44 | 68 |
| Example | pregeratinized | | | | |
| | starch) | | | | |
| | B(carmellose) | 26 | 24 | 22 | 19 |

According to Table 8, when using the α-1,4-glucan of sample 3, a tablet added with a larger amount of the α-1,4-glucan has a shorter disintegration time. However, when using the partially pregeratinized starch, the tablet has a longer disintegration time than that added with the α-1,4-glucan, and the tablet containing 50% or more of the partially pregeratinized starch had an extremely long disintegration time. Further, the tablet containing 75% or more of croscarmellose was cracked and had reduced hardness.

From the results, it was found that an increased addition ratio of an α-1,4-glucan does not lead to delay of disintegration and decreased hardness of a tablet.

### Example 5

Properties In Production by an Actual Machine

A tablet was produced by mixing sample 3 in Test Example 1 with ingredients in a composition as in Example 1, and tableting with a rotary tableting machine (manufactured by Kikusui Seisakusho Ltd., Cleanpress 19). For comparison, a tablet using microcrystalline cellulose (Avicel PH-101, manufactured by Asahi Kasei Corporation, sample name C) instead of the sample 3 was prepared. Tableting conditions were: 9 mm tablet diameter, 5.5 mm thickness and 1500 kgf of tableting pressure. The disintegration time in distilled water, the hardness, and the friability of the resultant tablets were measured. Results are listed in Table 9 below.

TABLE 9

| | | | hard- | |
|---|---|---|---|---|
| | | disintegration | ness | friability |
| | sample | time (second) | (kgf) | (%) |
| Example | 3(α-1,4-glucan) | 22 | 5.1 | 0.1 |
| Comparative | C(microcrystalline | 31 | 5.3 | 0.1 |
| Example | cellulose) | | | |

From the results, no effect on powder properties such as fluidability and adhesion upon tableting by adding the sample 3 was found. Further, the value of friability, which is an indicator of the degree of chip and wear during transport after preparation, was also problem-free in practice. From these results, it was found that an α-1, 4-glucan has no problem on tabletability and friability in production by an actual machine.

### Example 6

An Example Added as a Binder

A tablet was prepared according to a similar method as in Example 1 by using sample 4 or 5 obtained in Test Example 1 or amylose derived from potato starch obtained in Test Example 2 (sample D) in a composition ratio described in Table 10 below.

TABLE 10

| ingredient | parts by weight |
|---|---|
| ethenzamide | 400 parts |
| Avicel | 300 parts |
| Pharmacopoeia lactose | 265 parts |
| sample | 30 parts |
| magnesium stearate | 5 parts |
| total | 1000 parts |

The disintegration time in distilled water and the hardness of each of the resultant tablets were measured. Results are shown in Table 11 below.

TABLE 11

| | degree of | disintegration | |
|---|---|---|---|
| sample | polymerization | time (second) | hardness (kgf) |
| 5(α-1,4-glucan) | 1735 | 37 | 5.4 |
| 6(α-1,4-glucan) | 4818 | 41 | 6.3 |
| D (amylose derived | — | 75 | 5.1 |
| from potato starch) | | | |

From the results, it was found that there was a delay in disintegration time of a tablet using amylose derived from potato starch, compared to tablets using an α-1,4-glucan of a high degree of polymerization (samples 5 and 6).

### Example 7

Tablet Using a Mixture of α-1,4-Glucans

α-1,4-glucans of samples 5 and 3 obtained in Test Example 1 were mixed in three weight ratios of 5:95,10:90 and 30:70 to obtain samples (i), (ii) and (iii), respectively. By using samples (i) to (iii) and samples 3 and 5, respectively, in a composition ratio described in Table 12 below, tablets were prepared by a similar method as of Example 3.

TABLE 12

| ingredient | parts by weight |
|---|---|
| acetaminophen | 400 parts |
| sample | 600 parts |
| total | 1000 parts |

The disintegration time in distilled water and the hardness of each of the resultant tablets were measured. Results are shown in Table 13 below.

TABLE 13

| | ratio of sample | disintegration | |
|---|---|---|---|
| sample name | 5 to sample 3 | time (second) | hardness (kgf) |
| 3 | 0:100 | 24 | 4.3 |
| (i) | 5:95 | 25 | 5.6 |
| (ii) | 10:90 | 29 | 6.4 |
| (iii) | 30:70 | 33 | 7.1 |
| 5 | 100:0 | >3600 | 8.5 |

As a result, when mixing amylose having different molecular weights, the disintegration time and the hardness of the tablet were varied depending on the mixing ratio. When using sample 5 alone in this condition, the tablet turned into a gel and kept its tablet shape and was not disintegrated after one night. Such a change in disintegration time and hardness was found not to be proportional to the mixing ratio but to be a synergistic effect. From these results, it was found that the properties of a tablet are varied depending on the mixing ratio of α-1,4-glucans having different molecular weights, but the tablet still satisfies required hardness and disintegration time.

### Test Example 3

Carboxymethylation Of α-1,4-Glucan

100 g of sample 3 obtained in Example 1 was suspended in 1500 ml of methanol. To this was added 40 weight % of sodium hydroxide solution (230 ml) and 128 g of monochloroacetic acid, and reacted for 6 hours at 50° C. The resultant precipitate was collected by centrifugation, re-suspended in methanol, and then the precipitate was collected by centrifugation once again. After repeating this operation three times, the precipitate was heated and dried, ground, and passed through a 200-mesh sieve to give a carboxymethylated α-1,4-glucan.

### Example 8

Tablet Using A Carboxymethylated α-1,4-Glucan

The carboxymethylated α-1,4-glucan obtained in Test Example 3 was mixed with ingredients in the composition of Example land tableted using a desktop pressing machine under the same conditions as Example 1. The resultant tablet had a disintegration time of 18 seconds and a hardness of 5.2 kgf.

From this, it is shown that by carboxymethylating an α-1,4-glucan with a middle degree of polymerization, a tablet using it can have a shorter disintegration time while keeping its hardness.

### INDUSTRIAL APPLICABILITY

According to the present invention, a disintegrant for tablets, a binder for tablets and a binding-disintegrating agent for tablets having excellent disintegration and bondability containing an α-1,4-glucan are provided. A tablet containing an α-1,4-glucan is also provided. The disintegrant for tablets, the binder for tablets and the binding-disintegrating agent for tablets of the present invention can be used as an excipient.

1. A disintegrant for tablets consisting of an α-1,4-glucan having a degree of polymerization of not less than 180 and less than 1230 and a dispersity (weight average molecular weight “Mw”/number average molecular weight “Mn”) of not more than 1.25 or a modified product thereof.

2. The disintegrant according to claim 1, wherein said α-1,4-glucan is an enzymatically synthesized α-1,4-glucan.

3. The disintegrant according to claim 1, wherein said disintegrant is a modified product of said α-1,4-glucan, and said modification is a chemical modification selected from the group consisting of esterification, etherification and cross-linking.

4. A binder for tablets consisting of an α-1,4-glucan having a degree of polymerization of not less than 1230 and not more than 37000 and a dispersity of not more than 1.25 or a modified product thereof.

5. The binder according to claim 4, wherein said α-1,4-glucan is an enzymatically synthesized α-1,4-glucan.

6. The binder according to claim 4, wherein said binder is a modified product of said α-1,4-glucan, and said modification is a chemical modification selected from the group consisting of esterification, etherification and cross-linking.

7. A binding-disintegrating agent for tablets consisting of a low molecular weight α-1,4-glucan or a modified product thereof, and a high molecular weight α-1,4-glucan or a modified product thereof,
wherein said low molecular weight α-1,4-glucan has a degree of polymerization of not less than 180 and less than 1230 and a dispersity of not more than 1.25, and
wherein said high molecular weight α-1,4-glucan has a degree of polymerization of not less than 1230 and less than 37000 and a dispersity of not more than 1.25.

8. The binding-disintegrating agent according to claim 7, wherein said α-1,4-glucan is an enzymatically synthesized α-1,4-glucan.

9. The binding-disintegrating agent according to claim 7, wherein said binding-disintegrating agent is a modified product of said α-1,4-glucan, and said modification is a chemical modification selected from the group consisting of esterification, etherification and cross-linking.

10. The binding-disintegrating agent according to claim 7, wherein the weight ratio of said low molecular weight α-1,4-glucan or a modified product thereof to said high molecular weight α-1,4-glucan or a modified product thereof is 98:2 to 60:40.

11. The binding-disintegrating agent according to claim 7, wherein the weight ratio of said low molecular weight α-1,4-glucan or a modified product thereof to said high molecular weight α-1,4-glucan or a modified product thereof is 2:98 to 40:60.

12. A tablet comprising a low molecular weight α-1,4-glucan or a modified product thereof, and a high molecular weight α-1,4-glucan or a modified product thereof,
wherein said low molecular weight α-1,4-glucan has a degree of polymerization of not less than 180 and less than 1230 and a dispersity of not more than 1.25, and
wherein said high molecular weight α-1,4-glucan has a degree of polymerization of not less than 1230 and less than 37000 and a dispersity of not more than 1.25.
